(19) United States
(12) Plant Patent Application Publication
(10) Pub. No.: US 2003/0131389 P1
(43) Pub. Date: Jul. 10, 2003

(54) Ageratum plant named 'Agetis'

(76) Inventor: Monica M.A. Sanders, Grootebroek (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/045,978

(22) Filed: Jan. 10, 2002

(51) Int. Cl. A01H 5/00

(52) U.S. Cl. PLT/263

(57) ABSTRACT

A new Ageratum plant particularly distinguished by its large blue flower, early flowering, nicely branched plant habit and vigorous growth.

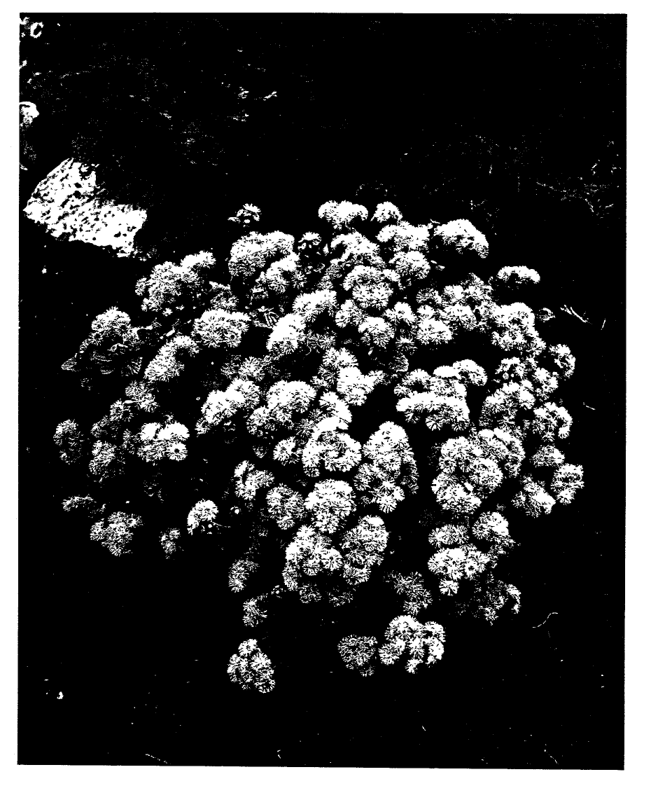

### BACKGROUND OF THE NEW PLANT

The present invention comprises a new distinct cultivar of Ageratum, botanically known as Ageratum houstonianum. The new cultivar is propagated from cuttings resulting from the cross of ‘Q343-2’ and ‘S570-2’. ‘Q343-2’ is a purple flowering Ageratum having a compact habit. ‘Q343-2’ is not commercially available and is not known by any synonyms. ‘S570-2’ is a blue and white flowering Ageratum having an upright habit. ‘S570-2’ is a selection from the variety ‘Southern Cross’.

Neither ‘Q342-2’ or ‘S570-2’ has been patented. As a result of this cross the present cultivar was created in 1997 in Enkhuizen, Netherlands and has been repeatedly asexually reproduced by cuttings in Enkhuizen, Netherlands, in Gilroy, USA, in Angers, France and in Sarrians, France over a three-year period. It has been found to retain its distinctive characteristics through successive propagations, and this novelty appears to be firmly fixed.

This new Ageratum plant is an annual in most climatical zones in the US, only in zones 9 and 10 is it a perennial plant.

### DESCRIPTION OF THE DRAWING

This new Ageratum plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Ageratum. The data which defines these characteristics were collected from asexual reproductions carried out in Enkhuizen, Netherlands. The plant history was taken on 20 weeks old plants, blossomed under natural light and artificial light in a greenhouse. Color readings were taken in the greenhouse under ambient light. Color references are primarily to the RHS Color Chart of The Royal Horticultural Society of London.

Differences between the new variety ‘Afetis’,
its parents and a similar cultivar

| | | | | |
|---|---|---|---|---|
| Character | ‘Agetis’ | ‘Q343-2’ | ‘S570-2’ | ‘R116’ |
| Flower color | Blue | Purple | Blue and | Blue |
| | | | white | |
| Size of | 16-21 mm | 6-9 mm | 16-21 mm | 7-9 mm |
| capitulum | | | | |
| Plant height | 30-40 cm | 20-25 cm | 25-35 cm | 20-25 cm |
| Branching | At every | At every | Not at | At every internode |
| | internode | internode | every | |
| | | | internode | |

The Plant:

Classification — Botanical.—Ageratum houstonianum.

Parentage — Female parent.—A seedling named ‘Q343-2’ is one of our seedlings from our Q-generation of plants bred in 1987 — Pollen parent.—A seedling named ‘S570-2’ is one of our seedlings from our S-generation of plants bred in 1997.

Growth habit.—Erect, well branched.

Plant height.—30-40 cm.

Spreading area of plant.—30-40 cm.

Growth rate.—Vigorous.

Strength.—Very good.

Branching character.—Freely branching and lateral branching at every node.

Blooming period.—Year round.

The Stem:

Diameter.—2-2.5 mm.

Shape.—Round.

Color.—145 B.

Anthocyan pigmentation.—Absent.

Length of internode.—30-42 mm, depending on the light where the plant is propagated.

Pubescence.—Slightly pubescent.

The Foliage:

Phyllotaxis.—Opposite, decussate.

Shape of blade.—Cordate.

Texture — Upper side.—Slightly pubescent — Lower side.—Slightly pubescent.

Venation.—Reticulate.

Leaf margin.—Crenate.

Leaf base.—Cordate.

Leaf apex.—Rounded.

Length.—27-63 mm.

Width.—30-68 mm.

Color — Upper side.—Dark green 137A — Lower side.—Medium green 143A.

Pubescence.—Some pubescence is present.

Length of petiole.—11-15 mm.

Color of petiole.—Light green 145C.

Petiole surface texture.—Slightly pubescent.

Inflorescense:

Inflorescense.—Compound umbel of capitula.

Length of peduncle.—18-35 mm.

Color of peduncle exposed to light.—RHS 152B.

Color of peduncle not exposed to light.—RHS 145A.

Length of pedicel.—4-11 mm.

Color of pedicel.—RHS 145A.

Number of capitula per inflorescense.—10-19.

Number of disc florets per capitulum.—80-120.

Number of bracts per capitulum.—23-25.

Length of bract.—4-5 mm.

Color of bracts.—RHS 143A.

Diameter of capitulum at beginning of flowering.—7-8 mm.

Color of capitulum at beginning of flowering.—RHS 083D.

Diameter of capitulum at full flowering.—16-21 mm.

Color of capitulum at full flowering.—RHS 086D.

Length of stigma and syle.—8-9 mm.

Color of stigma.—RHS 091A.

Fragrance.—No fragrance.

Lastingness of the bloom.—The capitula of one umbel open over a period of 5 to 6 weeks.

Seeds:

Seedset.—No seedset has been observed.

Roots:

Type of roots.—Fibrous. Roots start to grow on every part of the stem that contacts the soil, so not only at the nodes.

Physiological and ecological characteristics: Good tolerance to heat and cold, but no frost tolerance. Strong resistance to pests and diseases.

What is claimed is:

1. A new and distinct variety of Ageratum plant, substantially as herein illustrated and described, characterized particularly as to novelty by big blue flowers, which appear early on the plant, which produces no seeds, and has a growing habit that is erect, well branched and vigorous.
